(19) United States
(12) Patent Application Publication
(10) Pub. No.: US 2024/0108262 A1
(43) Pub. Date: Apr. 4, 2024

(54) HARASSMENT DETECTION APPARATUS AND METHOD

(71) Applicant: Sony Interactive Entertainment Inc., Tokyo (JP)

(72) Inventor: Jesus Lucas Barcias, London (GB)

(73) Assignee: Sony Interactive Entertainment Inc., Tokyo (JP)

(21) Appl. No.: 18/472,604

(22) Filed: Sep. 22, 2023

(30) Foreign Application Priority Data
Oct. 4, 2022 (GB) 2214582.5

(51) Int. Cl. A61B 5/16; A61B 5/024; A61B 5/0533; A61B 5/08

(52) U.S. Cl. CPC A61B 5/165; A61B 5/024; A61B 5/0533; A61B 5/0816

(57) ABSTRACT

A harassment detection apparatus includes: an executing unit configured to execute a session of a shared environment; an input unit configured to receive biometric data, the biometric data being associated with a plurality of users participating in the executed session of the shared environment; a generating unit configured to generate, based on at least a part of the biometric data, emotion data associated with the plurality of users, the emotion data comprising a valence value and/or an arousal value associated with each of the plurality of users; a detection unit configured to detect, responsive to at least a first part of the emotion data satisfying one or more of a first set of criteria, one or more first users associated with the at least first part of the emotion data; and a modifying unit configured to modify, responsive to the detection of the one or more first users, one or more aspects of the shared environment.

### BACKGROUND OF THE INVENTION

### Field of Invention

The present invention relates to a harassment detection apparatus and method.

### Description of the Prior Art

The rapid development in telecommunications technologies in recent years has enabled people to communicate with each other in a myriad of different ways, be it via a multi-player video game or virtual reality experience such as the metaverse (using Voice over Internet Protocol, for example), videoconferencing, chat rooms, instant messaging services, social media, telephony, or the like. These communication methods typically allow users to interact with each other through use of a shared environment (a virtual environment of a video game, a group chat in a social media application, a meeting in a videoconferencing application, or the like).

While such communication methods can be beneficial for user's wellbeing (by communicating with friends and family, for example), they are susceptible to being exploited by who use them for malicious purposes (such as harassment, defrauding others, or the like).

The present invention seeks to alleviate or mitigate this issue.

### SUMMARY OF THE INVENTION

In a first aspect, a harassment detection apparatus is provided in claim 1.

In another aspect, a harassment detection method is provided in claim 14.

Further respective aspects and features of the invention are defined in the appended claims.

A more complete appreciation of the disclosure and many of the attendant advantages thereof will be readily obtained as the same becomes better understood by reference to the following detailed description when considered in connection with the accompanying drawings, wherein:

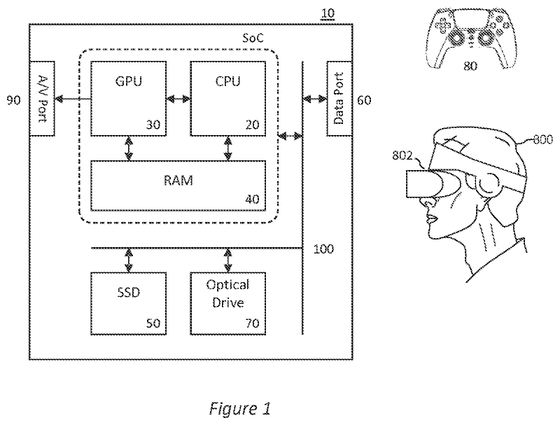

FIG. 1 schematically illustrates an entertainment system operable as a harassment detection apparatus according to embodiments of the present description;

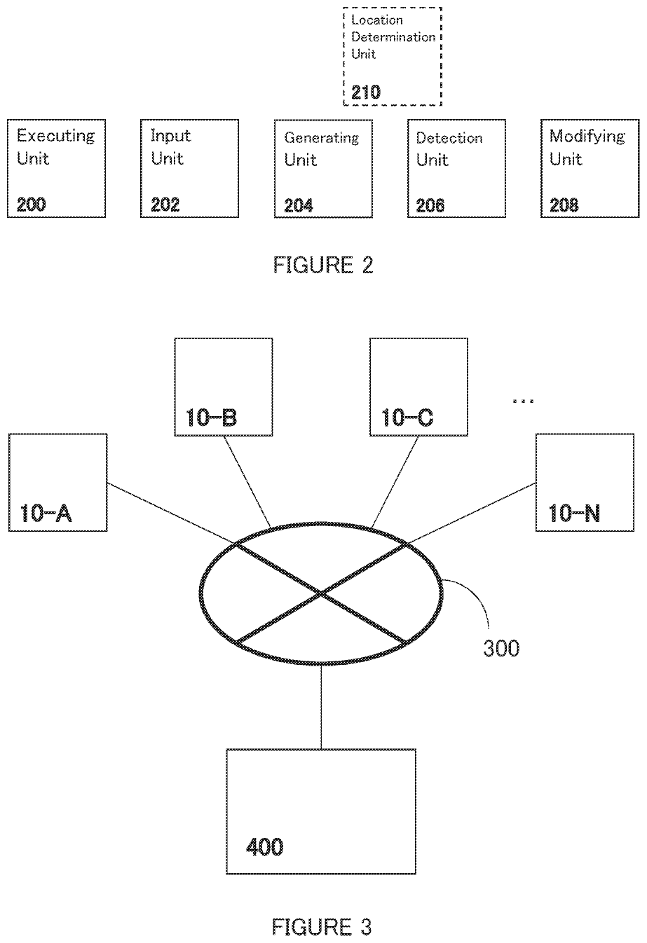

FIG. 2 is a schematically illustrates a harassment detection apparatus according to embodiments of the present description;

FIG. 3 schematically illustrates a network environment;

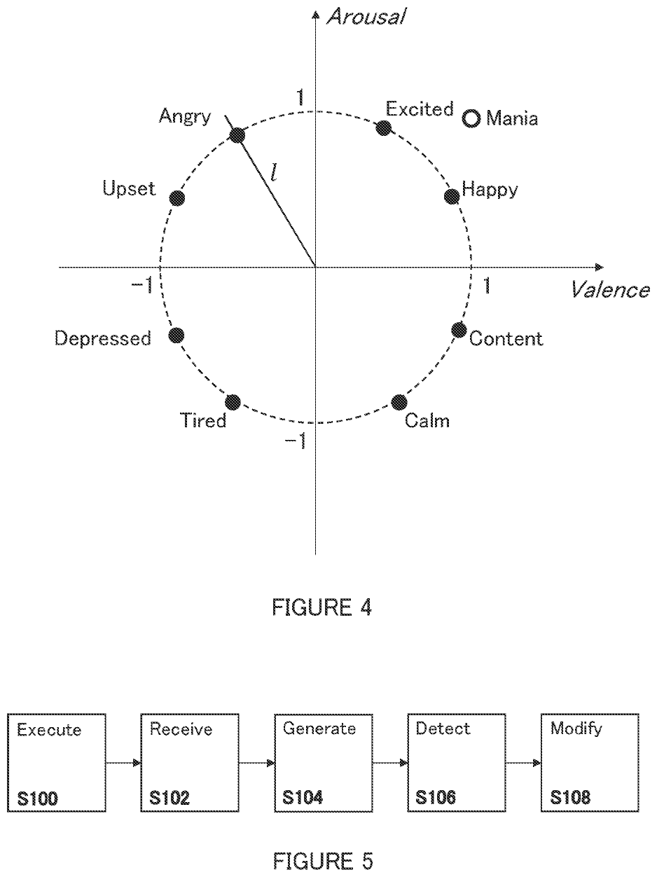

FIG. 4 schematically illustrates a valence and arousal chart; and

FIG. 5 schematically illustrates a harassment detection method according to embodiments of the present description.

### DESCRIPTION OF THE EMBODIMENTS

A harassment detection apparatus and method are disclosed. In the following description, a number of specific details are presented in order to provide a thorough understanding of the embodiments of the present invention. It will be apparent, however, to a person skilled in the art that these specific details need not be employed to practice the present invention. Conversely, specific details known to the person skilled in the art are omitted for the purposes of clarity where appropriate.

In an example embodiment of the present invention, an entertainment system is a non-limiting example of such a harassment detection apparatus.

Referring now to the drawings, wherein like reference numerals designate identical or corresponding parts throughout the several views, in FIG. 1 an example of an entertainment system 10 is a computer or console such as the Sony® PlayStation 5 ® (PS5).

The entertainment system 10 comprises a central processor 20. This may be a single or multi core processor, for example comprising eight cores as in the PS5. The entertainment system also comprises a graphical processing unit or GPU 30. The GPU can be physically separate to the CPU, or integrated with the CPU as a system on a chip (SoC) as in the PS5.

The entertainment device also comprises RAM 40, and may either have separate RAM for each of the CPU and GPU, or shared RAM as in the PS5. The or each RAM can be physically separate, or integrated as part of an SoC as in the PS5. Further storage is provided by a disk 50, either as an external or internal hard drive, or as an external solid state drive, or an internal solid state drive as in the PS5.

The entertainment device may transmit or receive data via one or more data ports 60, such as a USB port, Ethernet® port, WiFi® port, Bluetooth® port or similar, as appropriate. It may also optionally receive data via an optical drive 70.

Interaction with the system is typically provided using one or more handheld controllers 80, such as the DualSense® controller in the case of the PS5.

Audio/visual outputs from the entertainment device are typically provided through one or more A/V ports 90, or through one or more of the wired or wireless data ports 60.

Where components are not integrated, they may be connected as appropriate either by a dedicated data link or via a bus 100.

An example of a device for displaying images output by the entertainment system is a head mounted display ‘HMD’ 802, worn by a user 800.

As previously mentioned, certain users may wish to exploit shared environments for malicious purposes; to harass other users, for example. Harassment within shared environments can take on a myriad of forms. For example, harassment may occur via speech/audio, images/video or written text (expletives, rude hand/body gestures, threats, blackmailing, discrimination, doxing, or the like).

As will be appreciated by persons skilled in the art, the meaning of the term “shared environment” need not be limited to video game environments, but may also include other types of shared environments, such as social media, videoconferencing, chat rooms, instant messaging services, virtual reality experiences such as the metaverse, telephony, VoIP, and the like.

As will be appreciated by persons skilled in the art, harassment negatively impacts the wellbeing and safety of those to whom the harassment is directed. Given that people typically use shared environments as a way to improve their wellbeing through social interaction, it is desirable to detect harassment within shared environments, and subsequently enact measures to eliminate (or at least reduce the impact of) the harassment.

Current methods of harassment detection typically require the person experiencing harassment (hereinafter referred to as a “victim”) to transmit a report to a moderator (which may be a human or an automatized system). The report typically details the person whom the victim believes is harassing them (hereinafter referred to as a “harasser”), the type of harassment the victim is experiencing, and some data/metadata regarding the shared environment (timestamps of offending messages, session IDs of the video game in which the victim and harasser were playing, or the like). After review by the moderator, the harasser may be banned (either temporally or permanently) from the shared environment, or may be banned (either temporally or permanently) from interacting with the victim within the shared environment.

Because of this need for the victim to report the harassment they are experiencing (or have experienced), a certain proportion of harassment incidents may never be reported; the victim may find the processing of detailing the harassment to be negatively impacting their wellbeing further, and so instead chooses not to do so. This may result in certain harassers being able to carry on harassing other users, having faced no consequences for their previous harassment of others.

Thus, there is a need in the art for a harassment detection techniques that do not require the victim to report the harassment they are experiencing, which should thereby decrease the proportion of harassment incidents that never get reported and also improve the well-being and safety of users participating in shared environments.

### Harassment Detection Apparatus

The aforementioned problem of unreported harassment incidents can be alleviated or mitigated by implementing means to gather biometric data from users of a shared environment, and therefrom generate data indicating the emotions of the users. Such means would subsequently detect, using the emotion data, when a given user of the shared environment is experiencing negative emotions (sadness, fear, anxiety, or the like) and modify aspects of the shared environment (such as removing one or more other users, as a non-limiting example) in response thereto.

Accordingly, turning now to FIG. 2, in embodiments of the present description, a harassment detection apparatus comprises executing unit 200 configured to execute a session of a shared environment; input unit 202 configured to receive biometric data, the biometric data being associated with a plurality of users participating in the executed session of the shared environment; generating unit 204 configured to generate, based on at least a part of the biometric data, emotion data associated with the plurality of users, the emotion data comprising a valence value and/or an arousal value associated with each of the plurality of users; detection unit 206 configured to detect, responsive to at least a first part of the emotion data satisfying one or more of a first set of criteria, one or more first users associated with the at least first part of the emotion data; and modifying unit 208 configured to modify, responsive to the detection of the one or more first users, one or more aspects of the shared environment.

Shared environments are typically instantiated via a network such as LAN, WLAN, the Internet, P2P networks, or the like. FIG. 3 depicts a typical network environment for a multi-player video game, in which game server 400 is connected to a plurality of client devices (entertainment devices) 10-A to 10-N via network 300. Similar arrangements may be utilised with respect to social media platforms, videoconferencing, instant messaging, chat rooms, or the like. It should be noted that client devices need not only take the form of an entertainment device (such as a PS5), but may also include telephones, computers, televisions, or any other device capable of connecting to a network.

Therefore, and as will be appreciated by persons skilled in the art, a myriad of embodiments of the present description may arise within such a network environment. As a non-limiting example, a client device (such as entertainment device 10, 10-A, 10-B, and the like) may be made to operate (under suitable software instruction or even hardware adaptation) as a harassment detection apparatus according to embodiments of the present description. In the case of P2P networks, a client device may be the only suitable embodiment of the present description. In another non-limiting example, a server (such as game server 400) may be made to operate (under suitable software instruction or even hardware adaptation) as a harassment detection apparatus according to embodiments of the present description. In the case of multi-player video games, a server may be a more suitable embodiment of the present description. In yet another non-limiting example, a combination of client device and server may be made to operate as a harassment detection apparatus according to embodiments of the present description. In the case of multi-player video games, it may be prudent to make use of the advantages of distributed computing to ensure that the computational expenditure of harassment detection does not negatively impact gameplay (by increasing latency at the server, for example).

In essence, embodiments of the present description perform the detection of harassment by identifying a victim of harassment first, as opposed to identifying a harasser first. That is to say, the embodiments detect distress in a first user, rather than the computationally demanding task of detecting abusive behaviours in, from, or by a second user.

Harassment detection in this manner (that is, identifying a victim of harassment first as opposed to identifying a harasser first) is advantageous in that the number of false positive detections of harassment incidents may be reduced. For example, two people may be using expletives/displaying aggressive body language when interacting with each other. Were a “detect harassers first” approach to be used, then one (or even both) of the people may mistakenly be considered to be a harasser when in actuality they are friends joking with each other. Secondly, as noted above, detecting harassment may be computationally complex and requires monitoring on multiple fronts (e.g. audio, text, image, in-game behaviours, and the like), and moreover modes of harassment (e.g. abusive terms) differ with language and culture, and change all the time, requiring continuous updates to be current and effective. By contrast, the effect of harassment on an unfortunate victim tends to be universal and so embodiments of the present description advantageously make use of this simpler indication that effective harassment is occurring.

Accordingly, embodiments of the present description utilise a “detect victim first” approach to harassment detection. It should be noted that the terms “first users” and “victims” should be regarded as synonymous within the context of the present description by persons skilled in the art.

As a non-limiting example of the harassment detection apparatus in operation, consider a session of a multi-player video game in which a plurality of users are participating. During the game session, biometric data (such as heart rate, breathing rate, galvanic skin response, facial images, or the like) is obtained from the plurality of users. This is may be achieved by using, say, microphones, cameras, fitness tracking devices, mobile telephones, or the like, that are configured to communicate with entertainment device (games console) 10 via wired or wireless communication methods such as Bluetooth®, USB, WiFi®, or the like. The biometric data is received at input unit 202, and generating unit 204 subsequently generates emotion data (valence and/or arousal values) for each user therefrom. Alternatively or in addition, similarly controller 80 or HMD 802 may comprise biometric sensors such as one or more skin galvanic conduction sensors, or more or more cameras and/or microphones.

During gameplay, user A may start to intimidate or threaten user B. Because of this, user B may start to feel upset, afraid, worried, for example. As such, user B's biometric data may change (heart rate increases, breathing rate increases, facial images depict a frowning face, for example), leading to a change in user B's emotion data. Detection unit 206 may detect that user B is a victim of harassment when their emotion data (or changes in their emotion data) satisfies a first set of criteria that correspond to negative emotions (which shall be discussed later herein).

In response to detecting that user B is a victim of harassment, modifying unit 208 may modifying one or more aspects of the game session. Example modifications may include relocating user B's avatar within the video game environment, or relocating the other users' avatars within the video game environment such that they are at least a threshold distance away from user B's avatar, both of which may be particularly useful if the video game implements a so-called “proximity chat” feature where users can only communicate with each other if their avatars are within a threshold distance of each other. Other example modifications may include changing the communication settings of user B such that only those other users whose gaming profiles are connected with that of user B's gaming profile (alternatively put, those users are indicated as “friends” with user B) are permitted to communicate with user B, as opposed to all other users being permitted to do so. Shared environment modifications shall be discussed in more detail later herein.

The modifications to the shared environment seek to eliminate (or at least reduce the impact of) the harassment experienced by user B, thereby improving their safety and wellbeing while playing the video game.

### Shared Environments

In embodiments of the present description, executing unit 200 is configured to execute a session of a shared environment. In embodiments of the present description, executing unit 200 may be one or more CPUs (such as CPU 20, for example) and/or one or more GPUs (such as GPU 30, for example).

As previously mentioned, the term “shared environment” need not be limited to video game environments, but may also include other types of shared environments. As such the shared environment may be one of: i. a video game environment; ii. an online chat service (a chat room, for example); iii. an instant messaging service; iv. a videoconferencing service; and v. a social media platform.

It should be noted that the preceding examples are not exhaustive; persons skilled in the art will appreciate that types of shared environment other than those mentioned previously are considered within the scope of the present description.

### Biometric Data

In embodiments of the description, it is desirable to ascertain whether a given user participating in the shared environment is experiencing some form of harassment.

Therefore, in embodiments of the present description, input unit 202 is configured to receive biometric data, the biometric data being associated with a plurality of users participating in the executed session of the shared environment. In embodiments of the present description, input unit 202 may be one or more data ports, such as data port 60, USB ports, Ethernet® ports, WiFi® ports, Bluetooth® ports, or the like.

The biometric data obtained from the users during the executed session may provide an indication of each users' emotional state. This is because the human body typically exhibits a physiological response to emotions. For example, when experiencing fear, the human body typically responds by increasing heart rate, increasing breathing rate, sweating, and the like. Similarly, when a given user participating in a shared environment is experiencing harassment, their body will exhibit a physiological response to such harassment. The biometric data gathered from the given user during the executed session may comprise information regarding at least some aspects of their physiological response to harassment, which in turn may provide an indication of the emotional state of the user (subject to further processing discussed later herein). Hence, more generally, biometric data may be thought of as data indicative of a given user's physiological response to interactions occurring within the executed session of the shared environment.

The biometric data may comprise one or more of:
i. a galvanic skin response (changes in sweat gland activity and/or skin conductance);
ii. a heart rate;
iii. a breathing rate;
iv. a blink rate;
v. a metabolic rate;
vi. video data (one or more images of a given user's face and/or body);
vii. audio data (speech or other noises made by a given user); and
viii. one or more input signals (button presses from a given user's game controller).

It should be noted that the preceding examples are not exhaustive; persons skilled in the art will appreciate that types of biometric data other than those mentioned previously are considered within the scope of the present description.

The biometric data may be received from one or more of:
i. a fitness tracking device;
ii. a user input device (game controller, mouse, keyboard, or the like)
iii. a camera (standalone or comprised within a computer, head mounted display, TV, user input device, or the like); and
iv. a microphone (standalone or comprised within a computer, head mounted display, TV, user input device, or the like).

It should be noted that the preceding examples are not exhaustive; persons skilled in the art will appreciate that types of devices operable to obtain and transmit a given user's biometric data other than those mentioned previously are considered within the scope of the present description.

Optionally, input unit 202 may be configured to receive data comprising input signals from a plurality of input devices associated with the plurality of users. The advantages of doing so will be made apparent later herein.

It should be noted that the use of input signals as a source of biometric data is not one that must necessarily be obtained from all of the plurality of users; some users may provide such input signals as biometric data (either singly or in combination with other sources of biometric data), whereas some users may not. However, the aforementioned optional receiving of data comprising input signals at input unit 202 should be thought of as the receiving of all of the plurality of users input signals.

### Emotion Data

As previously mentioned, the biometric data gathered from a given user experiencing harassment during the executed session may comprise information regarding at least some aspects of their physiological response to harassment, which in turn may provide an indication of the emotional state of the user.

In order to ascertain the emotional state of the user, the biometric data may be used to generate a given user's valence and/or arousal values, which, in essence, respectively indicate the (un)pleasantness and/or the intensity of the emotion being experienced by the given user.

Therefore, in embodiments of the present description, generating unit 204 is configured to generate, based on at least a part of the biometric data, emotion data associated with the plurality of users, the emotion data comprising a valence value and/or an arousal value associated with each of the plurality of users. In embodiments of the present description, generating unit 204 may be one or more CPUs (such as CPU 20, for example) and/or one or more GPUs (such as GPU 30, for example).

The generated emotion data may provide an indication of a given user's emotional state, especially in the case where the emotion data comprises both valence and arousal values for the given user, as such values may be treated as a pair of coordinates to be plotted on a graph similar to that depicted in FIG. 4.

FIG. 4 depicts a valence and arousal chart onto which valence and arousal values associated with certain emotions have been plotted. Valence, v, and arousal, a, values are typically non-dimensional (that is, unitless) numbers within the range of −1≤v, a≤1. The values 1 and −1 therefore represent the extremes of (un)pleasantness and intensity of emotion. Interestingly, the valence and arousal values of commonly experienced emotions (such as those plotted with a black dot, Ω) typically follow the circumference of a circle (see dashed line) that is centred at the origin (which may represent an emotional state of ambivalence or indifference, for example) and has a circumference of 1. This is not necessarily true for all emotional states; certain rare (or less commonly experienced) emotional states, such as a very intense elation (plotted with a ring, o) experienced by some people during a manic episode, for example, may not fit this trend. However, given that this trend is typically followed by the more commonly experienced emotions, it may be beneficial to utilise this trend in the generation of emotion data. It should be noted that not all commonly experienced emotions are depicted in FIG. 4; persons skilled in the art will appreciate that other common emotions exist and may be plotted in similar manner to those depicted in FIG. 4. Line 1 shall be discussed later herein.

Generating unit 204 may generate emotion data from a given user's biometric data by implementing some form of predetermined algorithm (an equation, for example) which may take into account the different types of biometric data received from the given user. Certain types of biometric data may be more strongly correlated with valence than arousal, and vice versa. Therefore, those types of biometric data that are more correlated with valence (such as, video data and audio data, for example) may be used to determine the valence, and likewise those biometric data types that are more correlated with arousal (such as galvanic skin response and heart rate, for example) may be used to determine the arousal. Some types of biometric data may be correlated with both valence and arousal. For example, breathing rate may be positively correlated with arousal and negatively correlated with valence, which would result in the user's emotion data indicating calmness when the breathing rate is low, and an upset emotion when the breathing rate is high. The same could be said of a frequency of repeated input signals; a user may repeatedly press a button of a game controller while they experience anxiety, but not do so when they are calm.

It should be noted that the aforementioned correlations may only hold true in certain situations. People typically adopt a sitting positon when participating in shared environments. As such, the aforementioned correlations may hold true in these circumstances. However, if a certain user is, say, exercising while participating in a shared environment (for example, playing a video game based on physical activity, jogging while participating in a conference call, or the like), the aforementioned correlations may not hold true; the increased breathing rate of that certain user could not be relied upon as an indication that the user is upset. The same could be said for the type of video game being played (in the case where the shared environment is a video game environment); a horror game may cause certain users to feel scared/anxious/upset regardless of whether harassment is taking place or not. As such, and as will be appreciated by persons skilled in the art, other correlations would need to be devised which take into account the circumstances in which a given user is participating within the shared environment. Hence more generally the algorithm may optionally be specific to or calibrated for a particular environment or class of environment, and/or for particular levels or activities or classes thereof within the environment (e.g. being different during a death match game and during a matchmaking lobby where teams for the game are picked).

For the purposes of illustration only, the aforementioned correlations will be used in a non-limiting example of algorithmic emotion data generation. Equations 1 and 2 are example equations which may be used to calculate (generate) emotion data (valence and arousal values) from biometric data.

v=Qᵥ(c₁F+c₂A+c₃B+c₄I−D₁) Equation 1

a=Q_{d}(c₅S+c₆H+c₇B+c₈I−D₂) Equation 2

Qᵥ and Qₐ are scaling factors which ensure that the respective values of valence, v, and arousal, a lie in the range −1≤v, a≤1 (or at least ensure that the order of magnitude of v and a is 1). c₁ to c₈ are weighting coefficients for each type of biometric data, which may take on a positive or a negative value depending on the correlation between the respective type of biometric data and the valence/arousal value. F and A are quantities determined by respectively performing face detection on the video data and audio analysis on the audio data. For example, F may be the degree of curvature of the user's lips (indicating smiling or frowning), the degree of inclination of the eyebrows (indicating anger or surprise), or the like, and A may be an average pitch of the user's voice (higher pitch may indicate sadness), a quantity representing a timbre (tone colour) of the user's voice (whimpering has a timbre similar to that of a falsetto voice, whereas a normal voice is more full and resonant in sound), or the like. B is the breathing rate, I is the frequency of repeated input signals, S is skin conductance and H is heart rate. D₁ and D₂ are threshold (datum) values which the quantities c₁F+c₂A+c₃B+c₄I and c₅S+c₆H+c₇B+c₈I must respectively surpass so that v and a may be greater than zero (in the case, that the given user may be happy or excited, for example).

It should be noted that equations 1 and 2 (and any other equation which may be devised in order to generate emotion data from biometric data) may be tailored to each of the plurality of users. For example, where a given user does not provide breathing rate data, then equations 1 and 2 may be modified to discount or remove variables B, c₃ and c₇, and adjust (most likely reduce) the values of D₁ and D₂.

As previously mentioned, the valence and arousal values of commonly experienced emotions typically follow the circumference of a circle when plotted on a valence and arousal chart, the circle being centred at the origin and having a circumference of 1. This circle may therefore be expressed in equation 3 as:

v²+a²=1 Equation 3

This trend may be used in order to correct any errors which may arise when generating emotion data. For example, for a portion of biometric data, equation 1 may yield a valence value of 0.8, whereas equation 2 may yield an arousal value of 2.5 (which is erroneous as arousal values may only be between −1 and 1). This may be corrected by utilising equation 3 and the valence value of 0.8. Doing so yields an arousal value of ±0.36. Given that the erroneous arousal value was greater than zero, a corrected arousal value may be selected as 0.36.

Alternatively or in addition to using equations/algorithms, machine learning models may be used in order to generate emotion data from biometric data. This may be advantageous in that qualitative aspects of biometric data may be taken into account when generating the (typically quantitative) emotion data. These qualitative aspects may be the meaning (semantics) of the words spoken by a user (such words may be recognised using known speech recognition techniques), the cadence of a user's speech (which may not necessarily be qualitative, but the complex time-dependent nature of speech cadence may prove difficult to accurately take into account when using the aforementioned equations), determining the types of sounds uttered by the user (sobbing, screaming, whimpering, or the like), determining emotions from facial expressions, or the like. The use of machine learning models shall be discussed later herein.

In any case, the generated emotion data may be used to detect victims of harassment (and optionally harassers) within the shared environment.

### Detecting Victims (and Harassers)

As mentioned previously, it is desirable to detect the victims of harassment first in order to avoid (or at least reduce the occurrence of) false positive harassment detections.

In order to detect victims of harassment, the generated emotion data for each user may be evaluated against a set of criteria that correspond to negative emotions. Thus, a given user may be found to be a victim of harassment once their emotion data satisfies at least one of the criteria.

Therefore, in embodiments of the present description, detection unit 208 is configured to detect, responsive to at least a first part of the emotion data satisfying one or more of a first set of criteria, one or more first users associated with the at least first part of the emotion data. In embodiments of the present description, generating unit 204 may be one or more CPUs (such as CPU 20, for example) and/or one or more GPUs (such as GPU 30, for example).

Given that the first set of criteria (first criteria) are to be used to detect victims of harassment (first users), such first criteria should correspond to valence and arousal values typically associated with negative emotions. A graph similar to that depicted in FIG. 4 may be useful in the determination as to which valence and arousal values typically correspond to negative emotions. For example, persons skilled in the art would readily perceive that a valence value less than zero may serve as a suitable first criterion, as negative emotions typically have a negative valence value. It be should be noted that first criteria need not only specify valence and arousal values that satisfy some threshold value (valence being less than zero, for example) or some threshold range of values (arousal being between 0.2 and 0.5, for example), but may also specify changes in valence and arousal values (valence decrease of 0.4, for example), or even rates in these changes (arousal decrease of 0.3 in under 2 seconds, for example).

In any case, and more generally, a given one of the first set of criteria is such that valence and/or arousal values corresponding to negative emotions and/or negative changes in emotion would satisfy it.

As such, detection unit 206 may find that one or more users participating in the shared in environment are victims of harassment in the event that those one or more users' emotion data satisfies at least one of the first criteria. Consequently, detection unit 206 may detect harassers by detecting one or more second users that are different from the one or more first users. This detection of harassers, however, may be too crude in some instances. For example, if 50 people are participating within a shared environment, and one of those is found to be a victim of harassment, it may transpire that detection unit 206 finds the other 49 users to all be harassers. Thus, a more refined harasser detection may be needed in some instances. Such detection methods are discussed in the following paragraphs.

Optionally, detection unit 206 may be used to detect harassers in a similar manner. That is to say, the generated emotion data for each user may be evaluated against a set of criteria that corresponds to positive emotions, as harassers typically derive pleasure from harassing others. As such, detection unit 206 may be configured to detect, responsive to at least a second part of the emotion data satisfying one or more of a second set of criteria, one or more second users associated with the at least second part of the emotion data. The terms “second users” and “harassers” should be regarded as synonymous within the context of the present description by persons skilled in the art.

Given that the second set of criteria (second criteria) are to be used to detect harassers (second users), such second criteria should correspond to valence and arousal values typically associated with positive emotions. A graph similar to that depicted in FIG. 4 may be useful in the determination as to which valence and arousal values typically correspond to positive emotions. For example, persons skilled in the art would readily perceive that a valence value greater than zero may serve as a suitable second criterion, as positive emotions typically have a positive valence value. Similarly with first criteria, It be should be noted that second criteria need not only specify valence and arousal values that satisfy some threshold value or some threshold range of values but may also specify changes in valence and arousal values or even rates in these changes.

Turning back to FIG. 4, the line 1 serves as a potential demarcation between first and second sets of criteria (were both sets of criteria to be utilised). Given that harassment incidents typically arise through a user actively seeking to harass another user, harassers do not typically possess a low arousal value (which indicates a low intensity of emotion). Moreover, given that some forms of harassment can be quite aggressive/abusive, harassers may sometimes possess a negative valence value. As such, a more reliable detection of harassers may be carried out by creating second criteria that correspond to valence and arousal values which fall within the segment of the circle bounded by the positive valence axis and the line 1. Moreover, a more reliable detection of victims may be carried out by first criteria that correspond to valence and arousal values which fall within the segment of the circle bounded by the negative arousal axis and the line 1; victims do not typically possess positive valence values, and their arousal values typically do not exceed that associated with anger.

As a further option, should input unit 202 be configured to receive data comprising input signals from a plurality of input devices associated with the plurality of users, detection unit 206 may be configured to detect one or more input signals received from one or more of the second users within a threshold prior of time prior to and/or subsequent to the detection of the one or more first users. In doing so, detection unit 206 essentially performs a more refined detection of harassers. For example, subsequent to a victim being detected, several potential harassers may be detected. However, not all of these potential harassers may be a harasser. For example, some of the potential harassers may actually be engaged in an entertaining conversation with users other than the victim, but their emotion data has caused detection unit 206 to (erroneously) determine they had been harassing the victim (or another user). Thus, detection unit 206 may essentially filter out those potential harassers that in actuality are not harassers by taking into account the input signals received from the potential harassers. Should a given potential harasser be found to have provided an input signal within a threshold period of time prior to and/or subsequent to the victim detection, then that given potential harasser may be detected as the harasser, whereas those that had not done so (for example, the aforementioned entertaining conversation started after the threshold period of time subsequent to victim detection had elapsed) are not detected as the harasser.

Alternatively, harassers may be detected solely based on whether their input signals are received within a threshold period of time prior to and/or subsequent to the detection of the one or more first users. Accordingly, should input unit 202 be configured to receive data comprising input signals from a plurality of input devices associated with the plurality of users, detection unit 206 may be configured to: detect one or more input signals received within a threshold period of time prior to and/or subsequent to the detection of the one or more first users, and detect one or more second users associated with the detected input signals.

The threshold period of time in question may be predetermined, user-defined, dynamically adjustable during the executed session (in response to changes in emotion data, for example), or the like.

As will be appreciated by persons skilled in the art, the detection of harassers based on the timing of their input signals need not be restricted simply to such timings per se, but may also be based on the type, number, frequency, or any other aspect of those input signals which are received at input unit 202 within the threshold period of time prior to and/or subsequent to the detection of victims. For example, an input signal which causes an video game avatar to perform a taunting gesture may be considered more harassing than that which causes an avatar to perform a greeting (hand wave) gesture.

In any case, the victims (and optionally harassers) are detected by detection unit 206, and the shared environment may be modified in response.

In a variant embodiment, the detection may be refined further to reduce false positives as follows.

Whilst one or more users may be victims of harassment in the event that those one or more users' emotion data satisfies at least one of the first criteria, there may be other causes of (typically negative) emotion that satisfy the first criteria, such as for example losing the game, or getting killed, or being beaten to a piece of treasure, or the like. Hence optionally the system discounts emotion data satisfying the at least one of the first criteria if one or more relevant triggers of such emotion within the environment or gameplay have occurred within a threshold preceding period and/or distance of the user, or are clearly related to the user (such as their team conceding a goal).

By contrast harassment tends to be both opportunistic and takes some time to enact, and so will not have a consistent correlation with such in-game events. Of course, some potential harassment (such as teasing when the player loses) may be overlooked by this process, but sustained or repeated harassment, i.e. victimisation, can still be detected.

### Modifying Aspects of the Shared Environment

In embodiments of the present description, it is desirable to modify the shared environment once the victims (and optionally harassers) have been detected in order to eliminate (or at least reduce the impact of) the harassment experienced by victims, and thus improve user safety and wellbeing while participating in the shared environment.

Therefore, in embodiments of the present description, modifying unit 208 is configured to modify, responsive to the detection of the one or more first users, one or more aspects of the shared environment. In embodiments of the present description, modifying unit 208 may be one or more CPUs (such as CPU 20, for example) and/or one or more GPUs (such as GPU 30, for example).

As will be appreciated by persons skilled in the art, aspects of the shared environment that are to be modified by modifying unit 208 should be those that would reasonably be expected to affect an improvement in user (particularly victim) safety and wellbeing. As such, the aspects of the shared environment may be one or more of: i. a presence of one or more of the second users within the shared environment (banning harassers from participating within the shared environment either temporarily or permanently, for example); ii. an ability of one or more of the second users to provide one or more types of input signals to the shared environment (restricting harassers' usage of the shared environment either temporarily or permanently—allowing harassers to continue participating within the shared environment, but not allowing them to speak or send messages, for example); iii. a location of a given second user's avatar within the shared environment (should the shared environment be a video game environment, then the harassers' avatars may be relocated away from the victims' avatars); and iv. a location of a given first user's avatar within the shared environment (should the shared environment be a video game environment, then the victims' avatars may be relocated away from the harassers' avatars).

Optionally, modifying unit 208 may be configured to modify, responsive to the detection of the one or more second users, one or more aspects of the shared environment. Thus, modifying unit 208 would only carry out modifications to the shared environment when at least one victim and at least one harasser have been detected, techniques for harasser detection being discussed hereinbefore. This may provide a more targeted/refined modification to the shared environment, as the modifications carried out may be restricted to those which would only affect the detected victims' and/or harassers' participation within the shared environment, as opposed to modifications that affect every users' participation within the shared environment, the latter potentially arising in the case where modifying unit 208 modifies the shared environment only in response to the detection of victims (and every other user potentially being presumed a harasser).

As a further option, modifying unit 208 may be configured to modify one or more aspects of the shared environment in response to the detection of the one or more second users occurring within a threshold period of time prior to and/or subsequent to the detection of the one or more first users. As a non-limiting example, in response to a victim being detected, detecting unit 206 may detect several potential harassers by evaluating emotion data against the second criteria. However, not all of these potential harassers may be a harasser. As mentioned in a previous example, some of the potential harassers may actually be engaged in an entertaining conversation with users other than the victim, but their emotion data has caused detection unit 206 to (erroneously) determine that they had been harassing the victim (or another user), for example. Thus, detection unit 206 may essentially filter out those potential harassers that in actuality are not harassers by taking into account the timings of the victim and harasser detections. Similar bases for filtration may be whether such false harassers move with the victim, or for example are moving in response to each other and/or facing each other during their conversation. In other words, alternatively or in addition to temporal filters, and/or spatial filters (e.g. based on distance), directional and/or motion filters can optionally be used.

Should a given potential harasser be found to have been detected within a threshold period of time prior to and/or subsequent to the victim detection (or within equivalent thresholds for other bases of detection if used), then that given potential harasser may be detected as the harasser, whereas those that had not done so (for example, the aforementioned entertaining conversation started after the threshold period of time subsequent to victim detection had elapsed) are not detected as the harasser. Therefore, a more targeted/refined modification of the shared environment may be carried out by virtue of the refined harasser detection carried out by beforehand.

Where harasser detection is based on both emotion data evaluation and input signal timing (as discussed hereinbefore), modifying unit 208 may be configured to modify, based on the one or more detected input signals, one or more aspects of the shared environment, the detected input signals being those received from one or more of the second users within a threshold prior of time prior to and/or subsequent to the detection of the one or more first users. Again, a more targeted/refined modification of the shared environment may be carried out by virtue of the refined harasser detection carried out beforehand.

Given the possibility of false positives, optionally the modifications may be implemented in a sequence of mounting severity for the second user as successive harassment events are detected, either within a single session or over a plurality of sessions or a predetermined period. Where such modifications relate to mitigating the effect on one victim, the successive harassments may be specific to that victim. Meanwhile there such modifications relate to punishing or disenfranchising the second user, the successive harassments may be in relation to any victim. Consequently in such a scheme different forms of modification may occur at different rates of response to harassing behaviour.

### Avatar Locations

As noted previously herein, where embodiments of the present description are to be implemented as part of a video game (that is to say, where the shared environment is a video game environment), it may be beneficial to utilise the locations of users' avatars in order to determine which of the other users are harassers. This may provide another more refined harasser detection, as those users that are within a threshold distance from a victim are typically more likely to have been the harasser; users having an entertaining conversation on a mountain summit (and thus exhibiting positive emotions similar to those of a harasser) are not likely to have harassed a victim located on a riverbank.

Therefore, embodiments of the present description may comprise a location determination unit configured to determine a location of a plurality of avatars within the shared environment, each avatar being associated with a respective one of the plurality of users. Accordingly, detection unit 206 may be configured to: for a given avatar that is associated with a given first user, detect one or more avatars that are not associated with a given other first user located within a threshold distance from the given avatar, detect one or more second users associated with the one or more detected avatars. Moreover, modifying unit may be configured to modify, responsive to the detection of the one or more second users, one or more aspects of the shared environment.

The threshold distance in question may be predetermined, user-defined, dynamically adjustable during the executed session (in response to changes in emotion data, for example), or the like.

In essence, once a given victim has been detected, detection unit 208 detect whether a given other user is a harasser based on their avatar's proximity to the given victim's avatar.

### Generating Model

As mentioned previously, machine learning may be used in order to generate emotion data from biometric data. This may be advantageous in that qualitative aspects of biometric data may be taken into account when generating the (typically quantitative) emotion data, examples of such qualitative aspects being discussed hereinbefore. Therefore, in embodiments of the present description, generating unit 204 may comprise a generating model trained to generate the emotion data based on at least part of the biometric data.

As will be appreciated by persons skilled in the art, the generating model may be any type of machine learning model, neural network, or deep learning algorithm. As a non-limiting example of training, the generating model may be trained using training data comprising a plurality of types of biometric data and corresponding emotion (valence and arousal) data.

Optionally, the generating model may be trained using biometric data received during one or more previously executed sessions of the shared environment. As a non-limiting example, during those previously executed sessions, a conventional reporting of harassment may have been implemented. Subsequently, the generating model may be trained using the biometric data gathered during those previously executed sessions (previously received biometric data) and any data/metadata comprised within any harassment reports (victim user ID, harasser user ID, nature of harassment, or the like). The victims and/or harassers from these previous session may be identified through the harassment report data/metadata, and the biometric data of those victims and/or harassers may be used as training data (subject to any prior processing such as labelling). Alternatively or in addition to conventional reporting, emotion data that was generated during the previously executed sessions using any of the techniques discussed hereinbefore may be used.

Alternatively or in addition to training based on previously received biometric data, the generating model may be trained to generate the emotion data based on video data and/or audio data output from the shared environment. In such case, input unit 202 may be configured to receive the video data and/or audio data output from the shared environment. Such outputted video and/or audio data may comprise evidence of harassment occurring within the shared environment. As a non-limiting example, in a virtual reality environment (where users can typically manipulate the limbs of their avatars with a greater degree of freedom than that of conventional video games), a harasser may be causing their avatar to perform a rude gesture towards a victim. The victim's/harasser's/other users' point(s) of view of the video game environment (provided via their respective virtual cameras) may capture images/video frames of the harasser's avatar performing the rude gesture, and these images/video frames may be input to the generating model along with any emotion data that was generated during the session of the virtual reality environment using any of the techniques discussed hereinbefore, for example. The same can be said for any sounds (audio data) output from a virtual reality environment (or indeed any other type of shared environment). The generating model may then learn relationships between the bodily motions of avatars (or even the bodily motions of humans in the case of, say, a videoconferencing environment) and the emotion data, and/or relationships the speech/sounds of avatars/humans and the emotion data.

Optionally, the generating model may be trained using video data and/or audio data outputted during one or more previously executed sessions of the shared environment. This video and/or audio data may be used in similar manner to that discussed with respect to previously received biometric data, for example.

It will be appreciated that, as noted previously, different criteria may apply to different games or activities within a game, and hence similarly respective generating models may be trained or cloned and re-trained and used, for different games and/or different activities within a game.

In any case, the generating model, once trained, may provide a more comprehensive victim (and harasser) detection. This is because the generating model may be able to take into account more subtle and/or more qualitative aspects of human (or avatar) body language, speech, physiology, and the like into account when determining the emotions of users participating within a shared environment.

### Summary Embodiment(s)

Hence, in a summary embodiment of the present description a harassment detection apparatus comprises: executing unit 200 configured to execute a session of a shared environment; input unit 202 configured to receive biometric data, the biometric data being associated with a plurality of users participating in the executed session of the shared environment; generating unit 204 configured to generate, based on at least a part of the biometric data, emotion data associated with the plurality of users, the emotion data comprising a valence value and/or an arousal value associated with each of the plurality of users; detection unit 206 configured to detect, responsive to at least a first part of the emotion data satisfying one or more of a first set of criteria, one or more first users associated with the at least first part of the emotion data; and modifying unit 208 configured to modify, responsive to the detection of the one or more first users, one or more aspects of the shared environment, as described elsewhere herein.

It will be apparent to persons skilled in the art that variations in the aforementioned apparatus as described and claimed herein are considered within the scope of the present invention, including but not limited to that:
In an instance of the summary embodiment, detection unit 206 is configured to detect, responsive to at least a second part of the emotion data satisfying one or more of a second set of criteria, one or more second users associated with the at least second part of the emotion data; and modifying unit 208 is configured to modify, responsive to the detection of the one or more second users, one or more aspects of the shared environment, as described elsewhere herein;
In this instance, optionally modifying unit 208 is configured to modify one or more aspects of the shared environment in response to the detection of the one or more second users occurring within a threshold period of time prior to and/or subsequent to the detection of the one or more first users, as described elsewhere herein;
Similarly in this instance, optionally input unit 202 is configured to receive data comprising input signals from a plurality of input devices associated with the plurality of users; detection unit 206 is configured to detect one or more input signals received from one or more of the second users within a threshold prior of time prior to and/or subsequent to the detection of the one or more first users; and modifying unit 208 is configured to modify, based on the one or more detected input signals, one or more aspects of the shared environment, as described elsewhere herein;
In an instance of the summary embodiment, input unit 202 is configured to receive data comprising input signals from a plurality of input devices associated with the plurality of users; detection unit 206 is configured to: detect one or more input signals received within a threshold period of time prior to and/or subsequent to the detection of the one or more first users, and detect one or more second users associated with the detected input signals; and modifying unit 208 is configured to modify, responsive to the detection of the one or more second users, one or more aspects of the shared environment, as described elsewhere herein;
In an instance of the summary embodiment, the harassment detection apparatus comprises location determination unit 210 configured to determine a location of a plurality of avatars within the shared environment, each avatar being associated with a respective one of the plurality of users; wherein detection unit 206 is configured to: for a given avatar that is associated with a given first user, detect one or more avatars that are not associated with a given other first user located within a threshold distance from the given avatar, detect one or more second users associated with the one or more detected avatars; and modifying unit 208 is configured to modify, responsive to the detection of the one or more second users, one or more aspects of the shared environment, as described elsewhere herein;
In an instance of the summary embodiment, generating unit 204 comprises a generating model trained to generate the emotion data based on at least part of the biometric data, as described elsewhere herein;
In this instance, optionally the generating model is trained using biometric data received during one or more previously executed sessions of the shared environment, as described elsewhere herein;
Similarly in this instance, optionally input unit 202 is configured to receive video data and/or audio data output from the shared environment; and the generating model is trained to generate the emotion data based on the video data and/or audio data output from the shared environment, as described elsewhere herein;
In this instance, optionally the generating model is trained using video data and/or audio data outputted during one or more previously executed sessions of the shared environment, as described elsewhere herein;
In an instance of the summary embodiment, the biometric data comprises one or more selected from the list consisting of:
i. a galvanic skin response;
ii. a heart rate;
iii. a breathing rate;
iv. a blink rate;
v. a metabolic rate;
vi. video data;
vii. audio data; and
viii. one or more input signals, as described elsewhere herein;
In an instance of the summary embodiment, the biometric data is received from one or more selected from the list consisting of:
i. a fitness tracking device;
ii. a user input device;
iii. a camera; and iv. a microphone, as described elsewhere herein; and
In an instance of the summary embodiment, one or more of the aspects of the shared environment are one or more selected from the list consisting of:
i. a presence of one or more of the second users within the shared environment;
ii. an ability of one or more of the second users to provide one or more types of input signals to the shared environment;
iii. a location of a given second user's avatar within the shared environment; and
iv. a location of a given first user's avatar within the shared environment, as described elsewhere herein.

### Harassment Detection Method

Referring now to FIG. 5, a harassment detection method comprises the following steps: Step S100: executing a session of a shared environment, as described elsewhere herein. Step S102: receiving biometric data, the biometric data being associated with a plurality of users participating in the executed session of the shared environment, as described elsewhere herein. Step S104: generating, based on at least a part of the biometric data, emotion data associated with the plurality of users, the emotion data comprising a valence value and/or an arousal value associated with each of the plurality of users, as described elsewhere herein. Step S106: detecting, responsive to at least a first part of the emotion data satisfying one or more of a first set of criteria, one or more first users associated with the at least first part of the emotion data, as described elsewhere herein. Step S108: modifying, responsive to the detection of the one or more first users, one or more aspects of the shared environment.

It will be apparent to a person skilled in the art that variations in the above method corresponding to operation of the various embodiments of the apparatus as described and claimed herein are considered within the scope of the present invention.

It will be appreciated that the above methods may be carried out on conventional hardware (such as entertainment device 10) suitably adapted as applicable by software instruction or by the inclusion or substitution of dedicated hardware.

Thus the required adaptation to existing parts of a conventional equivalent device may be implemented in the form of a computer program product comprising processor implementable instructions stored on a non-transitory machine-readable medium such as a floppy disk, optical disk, hard disk, solid state disk, PROM, RAM, flash memory or any combination of these or other storage media, or realised in hardware as an ASIC (application specific integrated circuit) or an FPGA (field programmable gate array) or other configurable circuit suitable to use in adapting the conventional equivalent device. Separately, such a computer program may be transmitted via data signals on a network such as an Ethernet, a wireless network, the Internet, or any combination of these or other networks.

The foregoing discussion discloses and describes merely exemplary embodiments of the present invention. As will be understood by those skilled in the art, the present invention may be embodied in other specific forms without departing from the spirit or essential characteristics thereof. Accordingly, the disclosure of the present invention is intended to be illustrative, but not limiting of the scope of the invention, as well as other claims. The disclosure, including any readily discernible variants of the teachings herein, defines, in part, the scope of the foregoing claim terminology such that no inventive subject matter is dedicated to the public.

1. A harassment detection apparatus, comprising:
an executing unit configured to execute a session of a shared environment;
an input unit configured to receive biometric data, the biometric data being associated with a plurality of users participating in the executed session of the shared environment;
a generating unit configured to generate, based on at least a part of the biometric data, emotion data associated with the plurality of users, the emotion data comprising a valence value and/or an arousal value associated with each of the plurality of users;
a detection unit configured to detect, responsive to at least a first part of the emotion data satisfying one or more of a first set of criteria, one or more first users associated with the at least first part of the emotion data; and
a modifying unit configured to modify, responsive to the detection of the one or more first users, one or more aspects of the shared environment.

2. A harassment detection apparatus according to claim 1, wherein:
the detection unit is configured to detect, responsive to at least a second part of the emotion data satisfying one or more of a second set of criteria, one or more second users associated with the at least second part of the emotion data; and
the modifying unit is configured to modify, responsive to the detection of the one or more second users, one or more aspects of the shared environment.

3. A harassment detection apparatus according to claim 2, wherein the modifying unit is configured to modify one or more aspects of the shared environment in response to the detection of the one or more second users occurring within a threshold period of time prior to and/or subsequent to the detection of the one or more first users.

4. A harassment detection apparatus according to claim 2, wherein:
the input unit is configured to receive data comprising input signals from a plurality of input devices associated with the plurality of users;
the detection unit is configured to detect one or more input signals received from one or more of the second users within a threshold prior of time prior to and/or subsequent to the detection of the one or more first users; and
the modifying unit is configured to modify, based on the one or more detected input signals, one or more aspects of the shared environment.

5. A harassment detection apparatus according to claim 1, wherein:
the input unit is configured to receive data comprising input signals from a plurality of input devices associated with the plurality of users;
the detection unit is configured to:
detect one or more input signals received within a threshold period of time prior to and/or subsequent to the detection of the one or more first users, and
detect one or more second users associated with the detected input signals; and
the modifying unit is configured to modify, responsive to the detection of the one or more second users, one or more aspects of the shared environment.

6. A harassment detection apparatus according to claim 1, comprising a location determination unit configured to determine a location of a plurality of avatars within the shared environment, each avatar being associated with a respective one of the plurality of users;
wherein the detection unit is configured to:
for a given avatar that is associated with a given first user,
detect one or more avatars that are not associated with a given other first user located within a threshold distance from the given avatar,
detect one or more second users associated with the one or more detected avatars; and
the modifying unit is configured to modify, responsive to the detection of the one or more second users, one or more aspects of the shared environment.

7. A harassment detection apparatus according to claim 1, wherein generating unit comprises a generating model trained to generate the emotion data based on at least part of the biometric data.

8. A harassment detection apparatus according to claim 7, wherein the generating model is trained using biometric data received during one or more previously executed sessions of the shared environment.

9. A harassment detection apparatus according to claim 7, wherein:
the input unit is configured to receive video data and/or audio data output from the shared environment; and
the generating model is trained to generate the emotion data based on the video data and/or audio data output from the shared environment.

10. A harassment detection apparatus according to claim 9, wherein the generating model is trained using video data and/or audio data outputted during one or more previously executed sessions of the shared environment.

11. A harassment detection apparatus according to claim 1, wherein the biometric data comprises one or more of:
i. a galvanic skin response;
ii. a heart rate;
iii. a breathing rate;
iv. a blink rate;
v. a metabolic rate;
vi. video data;
vii. audio data; and
viii. one or more input signals.

12. A harassment detection apparatus according to claim 1, wherein the biometric data is received from one or more:
i. a fitness tracking device;
ii. a user input device;
iii. a camera; and
iv. a microphone.

13. A harassment detection apparatus according to claim 1, wherein one or more of the aspects of the shared environment are one or more of:
i. a presence of one or more of the second users within the shared environment;
ii. an ability of one or more of the second users to provide one or more types of input signals to the shared environment;
iii. a location of a given second user's avatar within the shared environment; and
iv. a location of a given first user's avatar within the shared environment.

14. A harassment detection method, comprising the steps of:
executing a session of a shared environment;
receiving biometric data, the biometric data being associated with a plurality of users participating in the executed session of the shared environment;
generating, based on at least a part of the biometric data, emotion data associated with the plurality of users, the emotion data comprising a valence value and/or an arousal value associated with each of the plurality of users;
detecting, responsive to at least a first part of the emotion data satisfying one or more of a first set of criteria, one or more first users associated with the at least first part of the emotion data; and
modifying, responsive to the detection of the one or more first users, one or more aspects of the shared environment.

15. A non-transitory, computer-readable storage medium having stored thereon a computer program comprising computer executable instructions adapted to cause a computer system to perform a harassment detection method, comprising the steps of:
executing a session of a shared environment;
receiving biometric data, the biometric data being associated with a plurality of users participating in the executed session of the shared environment;
generating, based on at least a part of the biometric data, emotion data associated with the plurality of users, the emotion data comprising a valence value and/or an arousal value associated with each of the plurality of users;
detecting, responsive to at least a first part of the emotion data satisfying one or more of a first set of criteria, one or more first users associated with the at least first part of the emotion data; and
modifying, responsive to the detection of the one or more first users, one or more aspects of the shared environment.

16. (canceled)
